(19) United States
(12) Patent Application Publication
(10) Pub. No.: US 2007/0276244 A1
(43) Pub. Date: Nov. 29, 2007

(54) Responsive medical diagnostic ultrasound imaging system and method

(75) Inventor: Lei Sui, Newcastle, WA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc.

(21) Appl. No.: 11/415,637

(22) Filed: May 2, 2006

(51) Int. Cl. A61B 8/00

(52) U.S. Cl. 600/443

(57) ABSTRACT

A medical diagnostic ultrasound imaging system is responsive to a user position. A sensor determines the position of the user. One or more components of the ultrasound imaging system move as a function of the sensed position. For example, the display and/or control panel of the ultrasound imaging system rotate or translate based on motion or a current position of the user. As the user leans or moves during an examination, at least a component of the ultrasound system compensates for the change in position. As another example, the ultrasound imaging system follows the user from one patient to another patient. As another example, the ultrasound imaging system packages itself. The user may move for purposes other than adjusting a position of the ultrasound imaging system or a component thereof, but the system or component moves to aid the user.

### BACKGROUND

This present embodiments relate to medical diagnostic ultrasound. In particular, the medical diagnostic ultrasound imaging system is response to the user.

During an ultrasound examination, a user may connect an appropriate ultrasound transducer probe to the ultrasound imaging system. The user then configures the ultrasound imaging system. The user configures with a control panel having a keyboard and/or other user input devices. A display of the ultrasound imaging system is typically positioned above the control panel. The control panel and/or the display may be manually adjustable in height, angle or other position.

While imaging, the user positions the transducer probe on or in a patient. The patient is typically in a bed adjacent to the ultrasound imaging system. However, the user may have to rotate, lean or move to properly position and/or maintain the position of the transducer probe relative to the patient. During the imaging, the user may also adjust the configuration of the ultrasound imaging system, requiring access to the control panel. During the imaging, the user may also view resulting images on the display. However, positioning the transducer probe may result in inconvenient, difficult or uncomfortable viewing or control of the ultrasound imaging system.

Ultrasound probes for insertion into a patient may include additional inputs. For example, a user pulls or pushes on guide wires to steer a catheter. The catheter includes an ultrasound transducer array for imaging. As another example, a transesophageal (TEE) ultrasound transducer probe may include a knob for rotating a transducer array. These further controls may require a user to position himself or herself adjacent a patient at a particular orientation, possibly making control or viewing of the ultrasound imaging system inconvenient, difficult or uncomfortable.

### BRIEF SUMMARY

By way of introduction, the preferred embodiments described below include methods, systems and computer readable media for a medical diagnostic ultrasound imaging system responsive to a user. A sensor determines the position of the user. One or more components of the ultrasound imaging system move as a function of the sensed position. For example, the display and/or control panel of the ultrasound imaging system rotate or translate based on motion or a current position of the user. As the user leans or moves during an examination, at least a component of the ultrasound system compensates for the change in position. As another example, the ultrasound imaging system follows the user from one patient to another patient. The user may move for purposes other than adjusting a position of the ultrasound imaging system or a component thereof, but the system or component moves to aid the user.

In a first aspect, a medical diagnostic ultrasound imaging system is responsive to a user. A sensor component connects with the ultrasound imaging system. The sensor component is operable, in part, to determine a position of the user. A motor is operable to move a system component as a function of the position of the user.

In a second aspect, a method is provided for a medical diagnostic ultrasound imaging system to respond to a user. A position of a user relative to the ultrasound imaging system is sensed. A user interface component of the ultrasound imaging system moves, substantially free of user force, as a function of a change in the position of the user relative to the ultrasound imaging system.

In a third aspect, a computer readable storage medium has stored therein data representing instructions executable by a programmed processor for responding to user position. The storage medium includes instructions for wirelessly determining with sensor signals the user position, and moving at least a component of the ultrasound imaging system relative to the user as a function of the user position.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a medical diagnostic ultrasound imaging system responsive to a user; and

FIG. 2 is a flow chart diagram of one embodiment of a method for a medical diagnostic ultrasound system to respond to a user change in position.

### DETAILED DESCRIPTION OF THE DRAWINGS AND PRESENTLY PREFERRED EMBODIMENTS

A position sensor on the ultrasound platform allows the platform to position itself automatically in favor of the user's operation. For example, the monitor tracks the user's position by adjusting height and/or angle. As another example, the platform follows the user from room to room or patient to patient. The ultrasound imaging system may be more mobile and user friendly.

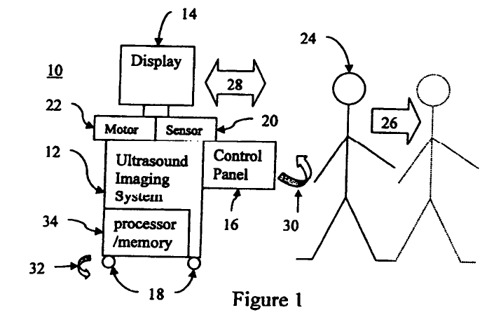

FIG. 1 shows a system 10 in which a medical diagnostic ultrasound imaging system 12 is responsive to a user 24. The user 24 is a person, such as a sonographer, physician or nurse, operating the medical diagnostic ultrasound imaging system 12. The user 24 controls the ultrasound imaging system 12 to examine a patient.

The ultrasound imaging system 12 includes a display 14, a control panel 16, wheels 18, a sensor 20, a motor 22, and a processor 34. Additional, different or fewer components may be provided. For example, the ultrasound imaging system 12 does not include the wheels 18. In one embodiment, the ultrasound imaging system 12 is a cart mounted system, but may be fixed in place, resting in place, handheld, portable or other type of ultrasound imaging system 12. As another example, the ultrasound imaging system 12 also includes a transducer probe or transducer connector, beamformer, detector, scan converter and/or other now known or later developed ultrasound imaging circuitry or processors.

The display 14 is a CRT, LCD, plasma, projector, printer, monitor or other now known or later developed display device. The display 14 is integrated into or positioned on a housing with the ultrasound imaging system 12. Alternatively, the display 14 is connected to or sits on the ultrasound imaging system 12. In other embodiments, the display 14 is spaced from the ultrasound imaging system 12.

The control panel 16 is a surface separate from, integrated on, or connected to the ultrasound imaging system 12. The control panel 16 includes a keyboard, trackball, touch pad, capacitive sensor, knob, dial, slider, button, mouse, combinations thereof or other now known or later developed user input devices. The control panel 16 is a single panel, but may be separated into a plurality of user input panels or locations. In one embodiment, the control panel 16 connects with, but extends from or is extendable from, the ultrasound imaging system 12. In other embodiments, such as a handheld or more integrated system 12, the control panel 16 is integrated on a housing of the ultrasound imaging system 12.

The wheels 18 are provided at each corner of the ultrasound imaging system 12. Any number of wheels 18 may be provided, such as three or four. Instead of wheels 18, the ultrasound imaging system 12 may include treads or other motive devices for moving or easing movement of the entire ultrasound imaging system 12.

The sensor 20 is active or passive, such as an infrared, heat, optical, vocal, sound, ultrasound, capacitive, electric field, electromagnetic, radar, radio frequency, laser, ranging or other now known or later developed sensor for detecting a position. The sensor 20 wirelessly determines the position of the user 24. For example, waves are transmitted and reflections received to determine the position. As another example, energy is received from the user 24 without transmission by the sensor 20, such as in optical sensing. The user 24 may have a reflective device, such as a sensor component of a material which interacts with the sensor 20. Another reflective device may include a harmonic generator or a radio frequency identification device.

The sensor 20 includes at least one component connected with the ultrasound imaging system 12. Sensor components include transmitters, receivers, communications devices, reflective devices or other sensor devices. In one embodiment, the entire sensor 20 connects with the ultrasound imaging system 12. In another embodiment, a portion of the sensor 20 is spaced away from the ultrasound imaging system 12. For example, the sensor 20 includes transmission and/or reception components spaced from the ultrasound imaging system 12. A signal receiver component of the sensor 20 is provided on the ultrasound imaging system 12 for receiving measurements or information from the other sensor components. Alternatively or additionally, a reflective device, such as the display 16 itself or a specially added device is a sensor component on the ultrasound imaging system 12. The reflective device allows sensing of the position of the ultrasound imaging system 12 so that a relative position between the user and the ultrasound imaging system 12 may be determined.

By positioning the transmission and/or reception components on, within, or at a defined position relative to or at a determined position relative to the ultrasound imaging system 12, the position of the user 24 relative to the ultrasound imaging system 12 or a component thereof is determined. In one embodiment, the sensor 20 is positioned on a main or primary housing of the ultrasound imaging system 12. Alternatively or additionally, the sensor 20 is positioned on another portion of the ultrasound imaging system 12, such as a moveable device (e.g., the display 14 and/or the control panel 16).

The sensor 20 is a single device or an array. For example, a plurality of sensors in an array is provided for triangulating a position of the user. As another example, a single camera or an array of optical sensors (e.g., CCD) scan or take a picture of a particular region relative to the ultrasound imaging system 12. The array may be in a same general location or distributed at different locations. Separate sensors 20 may be provided for controlling separate components of the ultrasound imaging system 12. Alternatively, a same sensor 20 controls just one or a plurality of components of the ultrasound imaging system 12.

The sensor 20 is operable, in part, to determine a position of the user. The position may be a distance along one axis, a position in a plane, or a three-dimensional position. The sensor 20 detects a particular part of the user 24, such as the head, a hand, or the torso. Alternatively, the sensor 20 detects the user 24 in general, such as detecting a nearest portion of the user 24 within the field of view of the sensor 20. The position is an absolute position, relative position, motion, or mere existence within a field of view.

The sensor 20 includes a circuit and/or processor for deriving the position from received information. Alternatively, the received information or partially processed information is provided to the processor 34 for determination of the position.

The motor 22 is a servo, electrical motor, a micro electromechanical device, a hydraulic pump or piston, combinations thereof or other now known or later developed device for moving one or more components. The motor 22 is positioned adjacent to or within the component of the ultrasound imaging system 12 to be moved. For example, the motor 22 is part of the mount or connector of the display 14 or control panel 16 with the ultrasound imaging system 12. As another example, the motor 22 is part of a drive chain for rotating the wheels 18.

A single motor 22 is provided for each system component to be moved. Alternatively, two or more motors 22 move a given system component. For example, four motors 22 cause rotation in a respective four wheels 18. As another example, more than one motor 22 allows a greater degree of freedom of movement for the display 14 or control panel 16, such as allowing rotation and tilting or translation. A common mount may be used for more than one system component, allowing one or more motors 22 to move two or more system components.

Under the control of the processor 34 with or without feedback from the motor 22, the motor 22 moves one or more system components as a function of the position of the user 24. For example, the display 14, control panel 16, control panel component (e.g., keyboard, or other user input), or combinations thereof are moved by the motor 22 in response to a position of the user 24. As the user 24 moves, a new position is determined. The system component is moved by the motor 22 in response to the new position. As another example, the wheels 18, tread, motive device or combinations thereof are activated by the motor 22 in response to the position of the user 24. As the user 24 changes locations, the ultrasound imaging system 12 follows or moves to a predetermined (e.g., docking) location. The sensor 20 or other sensors may be used for avoidance, such as preventing the ultrasound imaging system 12 from moving into the patient or other obstacle.

The motor 22 in conjunction with the sensor 20 causes the system component to alter position in response to user position. The user 24 may move for any reason, such as to examine a patient, position a transducer probe, view the display 16, input controls on the control panel 16, complete an examination, move to a different room, move to a different location relative to the patient, move to a different device, move to consult with another person or other reasons. The system component compensates, at least in part, for the change in position. For example, the display 14 rotates, tilts and/or translates for closer viewing or a more direct viewing angle relative to the user 24 or the user's head. As another example, the control panel 16 tilts, rotates and/or translates to a position more likely reachable by the user 24. The user motion is not applied to the system component, such as associated with pushing or pulling on the component to move the component. Instead, the component moves to accommodate the user.

The processor 34 is a general processor, control processor, digital signal processor, application specific integrated circuit, field programmable gate array, digital circuit, analog circuit, combinations thereof, or other now known or later developed device operable to process information. The processor 34 is part of the ultrasound imaging system 12, such as being a control processor of the ultrasound imaging system 12. In another embodiment, the processor 34 is a separate processor for operating the sensor 20 and the motor 22.

The system 10 includes a memory, such as memory for the processor 34, in the ultrasound system 12, or on a removable media. The memory is a computer readable storage medium having stored therein data representing instructions executable by a programmed processor, such as the processor 34.

The instructions implement the processes, methods and/or techniques discussed herein. The computer readable media may be a cache, buffer, RAM, removable media, hard drive or other computer readable storage media Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, filmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

The instructions cause the processor 34 to process information from the sensor 20 to control the motor 22. For example, the processor 34 accesses look-up tables or processes algorithms to determine a desired component location or motor operation for possible sensor outputs. The processor 34 adjusts the ultrasound imaging system's 12 or component's position, gesture or motion in favor of the user's 24 position, gesture or motion. Additional or different processes may be implemented by the processor 34 pursuant to the instructions, such as enabling or disabling the automated position control in response to user input on the control panel 16.

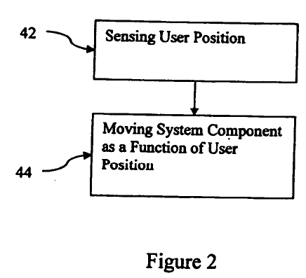

FIG. 2 shows one embodiment of a method for a medical diagnostic ultrasound imaging system to respond to a user. The method of FIG. 2 is implemented by the system 10 of FIG. 1 or a different system. Additional, different or fewer acts may be provided.

In act 42, a position of a user is sensed. The sensing uses acoustic, ultrasound, radar, electromagnetic, light, infrared or other energies. The energy is transmitted by the sensor or provided by the environment of the system. Wireless energy is used to determine the position of the user. The position is determined from received or sensed signals.

The position may be the existence of the user or an object within a field of view or a particular position (e.g., distance from the sensor) along a line, in an area or in a volume. The position information provided by the sensor is an absolute position or relative position.

The position is determined relative to the ultrasound imaging system or a component of the system, such as a display or control panel. Using a detected or known absolute position of the user or portion of the user and the system or a component of the system, the relative position is known or determined. Alternatively, the field of view and/or penetration depth of the sensor (e.g., sensor range) provides the position of the user relative to the sensor. With an assumed or known relationship of the sensor to the system, the relative position of the user to the system or component is known or determined.

The position of the user is based on the position of a particular portion of the user. For example, a position of the head of the user is determined. The sensor signals are filtered or processed to isolation information from the head of the user. A filter adapted to a generally circular or elliptical shape may sharpen or more likely identify a head shape. A position of a reflective device may be used to identify a particular portion of the user, such as sensing a greater reflectivity provided by a user's eyes or an objection on a surgical mask. Alternatively, the position corresponds to any portion of the user, a closest portion of the user or a general center of mass of the user, such as detected by capacitive sensing.

In act 44, at least one component of the ultrasound imaging system moves relative to the user. A motor or motors move the system or component. The motor operates pursuant to electrical, hydraulic, mechanical, or other forces. The movement is rotational and/or translational. The movement is in one or more degrees of freedom, such as rotating about or translating along a single axis or as rotating and/or translating along two or more axes. The motors move the system or component substantially free of user force. Substantially free provides for no user-applied force for movement of the system or component or for some forces, such as to initiate or influence movement.

The system or component moves as a function of the user position. For example, a user interface component of the ultrasound imaging system moves as a function of a change in the position of the user relative to the ultrasound imaging system or component. If the user moves away from or towards the patient, the system or component compensates or aligns to provide access to the current user position. If the user moves towards or away from the system or component, the system or component may move away or towards the user. If the user moves up or down relative to the system or component, the system or component may translate and/or rotate up or down. If the user moves to the side, the system or component may rotate and/or translate to the side. Combinations of the motions may be used. If the user moves into a field of view, the system or component may move to an orientation appropriate for the field of view. If the user moves from a field of view, the system or component may move to a pre-determined location or may move in a direction from which the user moved from the field of view.

The amount and speed of motion may be set, variable or disabled. For example, the amount and/or speed of motion may approximate or be proportional to the amount and/or speed of motion of the user. The system or component may have two or more set positions. The motion may place the system or component in the set position most appropriate for a current location of the user. Alternatively, a large or continuous range of positions is provided. Any relationship of user position to a position of the system or component may be used. Any threshold amount of user change in position may be used to cause a change in position of the system or component.

For movement of a component, the component moves closer to or points more directly towards the user position. Alternatively, the component moves away from the user position. The component, such as a user interface component (e.g., display or control panel), moves relative to at least a portion of the ultrasound imaging system. Rather than moving the entire system, the component moves relative to the system as a function of the user position relative to the system or the component. Alternatively, the movement of the system positions the component at a desired location.

In one embodiment, the display moves as a function of the user position. For example, the display is rotated to face the user more directly when the user's head changes position. Translation may be used additionally or alternatively. In another embodiment, the keyboard, control panel, display, or combinations thereof may move as a function of the user position, such as the position of the user in general, the user's head or the user's hand. In other embodiments, the entire system, such as the user interface components and other components of the ultrasound imaging system, move as a function of user position. For example, the system moves to maintain a desired distance or proximity to the user. As the user moves about a room or in a facility, the system follows the user. As another example, the system moves to a parking or out-of-the-way location when a user moves outside a particular range. The system can also change its shape or package itself into a compact structure to fit the space. Any of the embodiments may be used alone or in combination.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (We) claim:

1. A medical diagnostic ultrasound imaging system responsive to a user, the ultrasound imaging system comprising:
a sensor component connected with the ultrasound imaging system, the sensor component operable, in part, to determine a position of the user; and
a motor operable to move a system component as a function of the position of the user.

2. The ultrasound imaging system of claim 1 wherein the sensor component comprises a sensor.

3. The ultrasound imaging system of claim 2 wherein the sensor comprises an infrared sensor, optical sensor, ultrasound sensor, capacitive sensor, electromagnetic sensor or combinations thereof.

4. The ultrasound imaging system of claim 1 where the sensor component comprises a reflective device.

5. The ultrasound imaging system of claim 1 wherein the motor comprises a servo, electrical motor, or combinations thereof.

6. The ultrasound imaging system of claim 1 wherein the system component comprises a display, control panel, keyboard, user input, or combinations thereof.

7. The ultrasound imaging system of claim 1 wherein the system component comprises a wheel, tread, motive device or combinations thereof operable to change locations of the ultrasound imaging system as a function of the position of the user.

8. The ultrasound imaging system of claim 7 wherein the system component is operable to cause the ultrasound imaging system to follow the user.

9. The ultrasound imaging system of claim 1 wherein the motor in conjunction with the sensor component is operable to cause the system component to alter position in response to user motion for examining a patient and other than user motion applied to the system component.

10. A method for a medical diagnostic ultrasound imaging system to respond to a user, the method comprising:
sensing a position of a user relative to the ultrasound imaging system; and
moving, substantially free of user force, a user interface component of the ultrasound imaging system as a function of a change in the position of the user relative to the ultrasound imaging system.

11. The method of claim 10 wherein sensing comprises sensing a head position of the user relative to a display, and wherein moving comprises moving the display as a function of the head position.

12. The method of claim 10 wherein moving comprises moving a keyboard, control panel, display, or combinations thereof.

13. The method of claim 10 wherein moving comprises moving in response to user motion towards or away from a patient.

14. The method of claim 10 wherein moving comprises moving the user interface component relative to at least a portion of the ultrasound imaging system.

15. The method of claim 10 wherein moving comprises moving the user interface component and other components of the ultrasound imaging system.

16. In a computer readable storage medium having stored therein data representing instructions executable by a programmed processor for responding to user position, the storage medium comprising instructions for:
wirelessly determining with sensor signals the user position; and
moving at least a component of the ultrasound imaging system relative to the user as a function of the user position.

17. The instructions of claim 16 wherein wirelessly determining comprises determining in response to transmitted energy.

18. The instructions of claim 16 wherein moving comprises moving a display, control panel, keyboard, or combination thereof.

19. The instructions of claim 16 wherein moving comprises moving the component closer to the user position.

20. The instructions of claim 16 wherein determining the user position comprises determining a position of a portion of the user.
